(19) United States
(12) Patent Application Publication
(10) Pub. No.: US 2009/0182133 A1
(43) Pub. Date: Jul. 16, 2009

(54) BENZOPYRANONE DERIVATIVES AND THEIR USE AS ANTI-VIRAL AGENTS

(75) Inventors: Chum Mok Puah, Singapore (SG); Weiliang Zhu, Shanghai (CN); Jian Li, Shanghai (CN); Lili Chen, Shanghai (CN); Cheng Luo, Shanghai (CN); Gang Chen, Singapore (SG); Zhili Zuo, Singapore (SG); Xiaomin Luo, Shanghai (CN); Xu Shen, Shanghai (CN); Kaixian Chen, Shanghai (CN); Hualiang Jiang, Shanghai (CN)

Correspondence Address: K&L Gates LLP, P.O. Box 1135, CHICAGO, IL, 60690, US

(73) Assignees: Singapore Polytechnic; Shanghai Institute of Materia Medica Chinese Academy of Sciences

(21) Appl. No.: 12/159,570

(22) PCT Filed: Dec. 15, 2006
(86) PCT No.: PCT/SG2006/000392

§ 371 Date: Jan. 15, 2009

(30) Foreign Application Priority Data
Dec. 29, 2005 (CN) 200510132956.8
Sep. 18, 2006 (SG) 200606408-3

(51) Int. Cl. C07H 17/06; C07D 319/08

(52) U.S. Cl. 536/18.1; 549/365

(57) ABSTRACT

The invention relates to pharmaceutical compositions comprising benzopyranone derivatives for the treatment of Severe Acute Respiratory Syndrome (SARS).

### TECHNICAL FIELD

The present invention generally relates to benzopyranone derivatives and their use as anti-viral agents, and more specifically, to their use in medicine for the treatment of a patient suffering from Severe Acute Respiratory Syndrome (SARS), acute nasopharyngitis, or other related diseases.

### BACKGROUND

SARS Coronavirus belongs to the order of Nidovirales, family of Coronaviride and genus of Coronavirus. It is a type of globular, membrane enclosed positive RNA virus. There are more than 10 different types of the virus, which has been observed to cause respiratory and gastrointestinal diseases in humans and animals.

The Coronavirus may be divided into three groups: Group I and Group II being the viruses found mainly in mammals, such as humans; and Group III being found in avians.

The host range of the virus is limited and conditions for cell culture are very stringent. The optimum temperature for culturing human coronavirus ranges from 33° C. to 35° C. There are two antigenic types of human coronavirus: (i) OC43 and (ii) HCoV-229E. Both coronavirus types are the pathogens that cause upper-respiratory infection in humans and often cause acute nasopharyngitis (the common cold), which happens mainly in winter and early spring. The incubation period is from two to five days and the symptoms can last six to seven days. The major clinical symptoms include discomfort, rhinitis, headache, sore throat, cough, high fever, loss of voice, aches in chest and abdomen, etc.

The coronaviruses can also suddenly induce child asthma and, occasionally, aggravate adult chronic bronchitis.

Different countries have different rates of positive antibodies. In China, the rate of positive antibodies for coronaviruses is between 30% and 60%. In the former USSR, the rate of positive antibodies for coronaviruses was between 53% and 97%. In Washington D.C., serological epidemiological research based on four consecutive years show that 10% to 24% of the cases of coronaviral infection are attributed to upper-respiratory infections. In a family-based check in Michigan, it was found that coronavirus can infect the following age groups: 0 to 4 years old is 29.2%, above 40 years is 22% with the 15 the 19 years old group being the highest.

Coronavirus HCoV-229E is one type of common cold, which is responsible for about 30% of acute nasopharyngitis in humans.

Contagious atypical pneumonia, also called Severe Acute Respiratory Syndrome (SARS), is a disease caused by a new type of coronavirus. This virus does not belong to any type of the virus mentioned above. It can be spread via aerosols, contact with faeces or urine and many other routes. SARS is an acute disease that is highly contagious and results in a high mortality rate. The clinical symptoms include acute occurrence of initial symptoms of fever (>38° C.) such as chills, headaches, aches in joints and muscles, loss of energy, diarrhoea, and in some more serious cases, the rate of respiration accelerates with difficulties in breathing. For patients who are seriously affected with SARS, the symptoms can last for 18 to 23 days, but they may last longer.

Since November 2002, SARS began as an epidemic disease in Asia and soon spread all over the world. 90% of affected patients can recover fully from SARS and the death rate is about 10%. On 16 Apr. 2003, the World Health Organisation declared that the pathogen of SARS is a type of new coronavirus and formally named it as “SARS coronavirus”. SARS coronavirus is a single-stranded positive RNA virus, the replication of which bypasses DNA intermediate, using a standard codon.

It was discovered from research that a protein called 3CL proteinase plays an essential regulatory role in the viral life cycles of both SARS and viruses associated with acute nasopharyngitis. The virus can only complete its transcription and replication after the polyproteins expressed by the virus are cleaved by the 3CL proteinase. Hence, the 3CL proteinase is an ideal target for drug discovery.

Studies on 3CL proteinase have become the leading indicator for developing drugs to treat acute nasopharyngitis and SARS. If the activity of the 3CL proteinase can be effectively inhibited, the replication of the virus inside the body will be prevented, thereby treating acute nasopharyngitis or SARS.

There is a need to provide an effective anti-viral treatment for treating acute nasopharyngitis in a patient or for treating infection caused by SARS coronavirus.

There is a need to provide a medicine for at least ameliorating the symptoms associated with acute nasopharyngitis, or infection caused by SARS coronavirus, in a patient.

There is a need to provide active compounds for use in a medicament, which are relatively easy to synthesise.

There is a need to provide active compounds for use in a medicament, which have sufficiently low toxicity such that they are not harmful to humans.

There is a need to provide active compounds which are capable of inhibiting the transcription or replication of 3CL proteinase, and in particular, to inhibit 3CL proteinase (3CLMpro) of SARS virus and HCoV-229E.

### SUMMARY

According to a first aspect of the invention, there is provided a compound represented by the following formula (I):

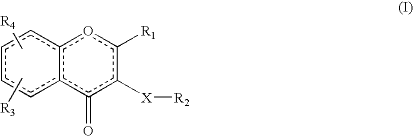

or a pharmaceutically acceptable salt thereof, wherein

[symbol] is an optional double bond;

X is independently selected from oxygen (O), nitrogen (N) and sulfur (S);

R₁ is an optionally substituted aromatic group;

R₂ is selected from the group consisting of straight or branched alkyl, alkenyl, alkynyl, cycloalkyl, lower cycloalkenyl, heterocyclic groups, aromatic groups, heteroaromatic groups, aralkyls, and glycosyl; and

R₃ and R₄ are independently selected from the group consisting of hydrogen (H), hydroxyl, nitro, amino, cyano, imdide, thiol, alkyl, alkenyl, alkynyl, alkoxy, haloalkyl, optionally substituted cycloalkyl, optionally substituted cycloalkenyl, aralkyl, and glycosyl.

In one embodiment R₂ is selected from the group consisting of lower alkyl, lower alkenyl, lower alkynyl, lower cycloalkyl, and lower cycloalkenyl.

In one embodiment R₃ and R₄ are independently selected from the group consisting of lower alkyl, lower alkenyl, lower alkynyl and lower alkoxy.

According to a second aspect, there is provided a pharmaceutical composition comprising one or more compounds of formula (I) as defined above, in admixture with a pharmaceutically acceptable carrier.

According to a third aspect, there is provided a method of treating or ameliorating the symptoms associated with Severe Acute Respiratory Syndrome (SARS) or acute nasopharyngitis (common cold virus) in a patient, the method comprising the step of administering to the patient, a pharmaceutical composition comprising one or more compounds of formula (I) as defined above in admixture with a pharmaceutically acceptable carrier.

According to a fourth aspect, there is provided a compound of formula (I) for use in medicine. In one embodiment, the compound is used to treat or inhibit the symptoms associated with Severe Acute Respiratory Syndrome (SARS) or common cold virus in a patient.

According to a fifth aspect, there is provided use of a compound of formula (I) in the manufacture of a medicament for treating or inhibiting the symptoms associated with Severe Acute Respiratory Syndrome (SARS) in a patient.

According to a sixth aspect, there is provided use of a compound of formula (I) in the manufacture of a medicament for treating or inhibiting the symptoms associated with acute nasopharyngitis in a patient.

According to a seventh aspect, there is provided a method of inhibiting the transcription or replication of 3CL proteinase, the method comprising the step of introducing, to the 3CL proteinase, a compound of formula (I) as defined in the first aspect.

In one embodiment, the patient is a mammal. The patient may be a human.

According to a eighth aspect, there is provided a kit comprising a pharmaceutical composition comprising one or more compounds of formula (I) as defined above in admixture with a pharmaceutically acceptable carrier, and instructions for administering the pharmaceutical composition to a patient to thereby treat or inhibit the symptoms associated with Severe Acute Respiratory Syndrome (SARS) or acute nasopharyngitis virus.

### DEFINITIONS

The following words and terms used herein shall have the meaning indicated:

The following are some definitions that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “alkyl group” includes within its meaning monovalent (“alkyl”) and divalent (“alkylene”), straight chain or branched chain, saturated or aliphatic groups having from 1 to 10 carbon atoms or from 1 to 6 carbon atoms or from 1 to 4 carbon atoms, eg, 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “lower alkyl” means an alkyl group having from 1 to 6 carbon atoms or from 1 to 4 carbon atoms, eg, 1, 2, 3, 4, 5, or 6 carbon atoms.

The term “alkenyl group” includes within its meaning monovalent (“alkenyl”) and divalent (“alkenylene”), straight or branched chain unsaturated aliphatic hydrocarbon groups having from 2 to 10 carbon atoms, or from 2 to 6 carbon atoms or from 2 to 4 carbon atoms, eg, 2, 3, 4, 5, 6, 7, 8, 9, or 10 carbon atoms and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “lower alkenyl” means an alkenyl group having from 2 to 6 carbon atoms or from 2 to 4 carbon atoms, eg, 2, 3, 4, 5, or 6 carbon atoms.

The term “alkynyl group” as used herein includes within its meaning monovalent (“alkynyl”) and divalent (“alkynylene”) straight or branched chain unsaturated aliphatic hydrocarbon groups having from 2 to ˜10 carbon atoms, or from 2 to 6 carbon atoms, or from 2 to 4, and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “lower alkynyl” means an alkynyl group having from 2 to 6 carbon atoms or from 2 to 4 carbon atoms, eg, 2, 3, 4, 5, or 6 carbon atoms.

The term “hydroxyl” is intended to mean the radical —OH.

The term “thiol” means —SH.

The term “cyano” means —CN.

The term “nitro” means —NO₂.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms or from 3 to 6 carbon atoms, eg, 3, 4, 5, 6, 7, 8, 9, or 10 carbon atoms. Examples of cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like.

The term “lower cycloalkyl” means a cycloalkyl group having from 3 to 6 carbon atoms or from 3 to 4 carbon atoms, eg, 3, 4, 5, or 6 carbon atoms.

The term “cycloalkenyl” as used herein, refers to cyclic unsaturated aliphatic groups and includes within its meaning monovalent (“cycloalkenyl”) and divalent (“cycloalkenylene”), monocyclic, bicyclic, polycyclic or fused polycyclic hydrocarbon radicals having from 3 to 10 carbon atoms, or from 3 to 6 carbon atoms and having at least one double bond, of either E, Z, cis or trans stereochemistry where applicable, anywhere in the alkyl chain. Examples of cycloalkenyl groups include but are not limited to cyclopropenyl, cyclopentenyl, cyclohexenyl, and the like.

The term “lower cycloalkenyl” means a cycloalkenyl group having from 3 to 6 carbon atoms or from 3 to 4 carbon atoms, eg, 3, 4, 5, or 6 carbon atoms.

The term “heterocyclic” is herein defined to mean a ring of carbon atoms containing at least one hetero atom, and further the ring may be saturated or unsaturated. Hence, the term includes within its meaning monovalent (“heterocycloalkyl”) and divalent (“heterocycloalkylene”), saturated, monocyclic, bicyclic, polycyclic or fused hydrocarbon radicals having from 3 to 10 ring atoms wherein 1 to 5 ring atoms are heteroatoms selected from O, N, NH, or S. Examples include pyrrolidinyl, piperidinyl, quinuclidinyl, azetidinyl, morpholinyl, tetrahydrothiophenyl, tetrahydrofuranyl, tetrahydropyranyl, and the like. The term heterocyclic as used herein also includes within its meaning monovalent (“heterocycloalkenyl”) and divalent (“heterocycloalkenylene”), saturated, monocyclic, bicyclic, polycyclic or fused polycyclic hydrocarbon radicals having from 3 to 10 ring atoms and having at least 1 double bond, wherein from 1 to 5 ring atoms are heteroatoms selected from O, N, NH or S.

The term “heteroaromatic group” and variants such as “heteroaryl” or “heteroarylene” as used herein, includes within its meaning monovalent (“heteroaryl”) and divalent (“heteroarylene”), single, polynuclear, conjugated and fused aromatic radicals having 6 to 20 atoms wherein 1 to 6 atoms are heteroatoms selected from O, N, NH and S. Examples of such groups include pyridyl, 2,2′-bipyridyl, phenanthrolinyl, quinolinyl, thiophenyl, and the like.

The term “glycosyl” is to be understood as meaning monosaccharide, disaccharide, trisaccharide and oligosaccharide radicals, preferably monosaccharides, disaccharides and trisaccharides and their analogues or derivatives. Exemplary glycosyls include glucopyranosyl, galactopyranosyl, mannopyranosyl, glucofurnaosyl, ribofuranosyl, arabinopyranosyl, lyxopyranosyl or D-glycero-D-glucoheptopyranosyl, maltosyl, maltotriosyl, maltotetraosyl, lactosyl, cellobiosyl, melibiosyl or 6-O-α- or β-ribofuranosyl)gluocopyranosyl, 2-acetylamido-2-deoxyglucopyranosyl, 2-amino-2-deoxyglucopyranosyl, 2-caproylamido-2-deoxyglucopyranosyl, 2-lauroylamido-2-deoxyglucopyranosyl, 2-myristoylamido-2-deoxyglucopyranosyl, 2-palmitoylamido-2-deoxyglucopyranosyl, 2-stearoylamido-2-deoxyglucopyranosyl, 4-azido-4-deoxyglucopyranosyl, 4-stearoylamido-4-deoxyglucopyranosyl, 4-dodecoylamido-4-deoxyglucopyranosyl, 6-hexadecanoylamido-6-deoxyglucopyranosyl, 2,6-diamino-2,6-dideoxyglucopyranosyl, 6,6′-diamino-6,6′-dideoxymaltosyl, 6-amino-6,6′-dideoxylactosyl, 6-deoxymannopyranosyl, 2-deoxyribofuranosyl, fucosyl, 5-fluoro-5-deoxyribofuranosyl, 6-O-methylglucopyranosyl, 6-deoxy-6-thioglucopyranosyl and 3-deoxy-3-nitrogalactopyranosyl.

The term “halogen” or variants such as “halide” or “halo” as used herein refers to fluorine, chlorine, bromine and iodine.

The term “heteroatom” or variants such as “hetero-” as used herein refers to O, N, NH and S.

The term “alkoxy” as used herein refers to straight chain or branched alkyloxy groups having from 1 to 10, or from 1 to 6 carbon atoms. Examples include methoxy, ethoxy, n-propoxy, isopropoxy, tert-butoxy, and the like.

The term “lower alkoxy” means an alkoxy group having from 1 to 6 carbon atoms or from 1 to 4 carbon atoms, eg, 1, 2, 3, 4, 5, or 6 carbon atoms.

The term “amino” as used herein refers to groups of the form —NRaRb wherein Ra and Rb are individually selected from the group including but not limited to hydrogen, optionally substituted alkyl, optionally substituted alkenyl, optionally substituted alkynyl, and optionally substituted aryl groups.

The term “aromatic group”, or variants such as “aryl” or “arylene” as used herein refers to monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like.

The term “aralkyl” as used herein, includes within its meaning monovalent (“aryl”) and divalent (“arylene”), single, polynuclear, conjugated and fused aromatic hydrocarbon radicals attached to divalent, saturated, straight and branched chain alkylene radicals.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, and isocyanate.

The present invention includes within its scope all isomeric forms of the compounds disclosed herein, including all diastereomeric isomers, racemates and enantiomers. Thus, formulae (I) should be understood to include, for example, E, Z, cis, trans, (R), (S), (L), (D), (+), and/or (−) forms of the compounds, as appropriate in each case.

In the context of this invention the term “administering” and variations of that term including “administer” and “administration”, includes contacting, applying, delivering or providing a compound or composition of the invention to an organism, or a surface by any appropriate means.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (eg. sheep, cows, horses., donkeys, pigs), laboratory test animals (eg. rabbits, mice, rats, guinea pigs, hamsters), companion animals (eg. cats, dogs) and captive wild animals (eg. foxes, deer, dingoes).

“Mammal” refers to any animal classified as a mammal, including humans, domestic and farm animals, and zoo, sports, and pet companion animals such as a household pet and other domesticated animal such as, but not limited to, cattle, sheep, ferrets, swine, horses, poultry, rabbits, goats, dogs, cats and the like. Preferred companion animals are dogs and cats. Preferably, the mammal is human.

As used herein, the term “pharmaceutically acceptable salt” refers to those salts which retain the biological effectiveness and properties of the active compound of the antimicrobial composition; which are not otherwise undesirable. A thorough discussion of pharmaceutically acceptable salts is available in Remington's Pharmaceutical Sciences (Mack Pub. Co., N.J. 1991).

As used herein, the term “SARS virus” refers to a pathogen (infectious agent) generally recognized as causing, or being associated with, Severe Acute Respiratory Syndrome (SARS) infection. SARS infection includes symptomatic and asymptomatic infections, and symptoms may range from severe forms of respiratory disease to milder flu-like or atypical presentations such as fever, myalgia, lethargy, gastrointestinal symptoms, cough, sore throat and other non-specific symptoms. Shortness of breath may occur later. The term includes “SARS-coronavirus” (“SARS-CoV”) and “SARS-CoV-like viruses”. The term further includes the naturally-occurring form of the pathogen (e.g., wild-type); naturally-occurring variants of the pathogen; and variants generated in the laboratory, including variants generated by selection, variants generated by chemical modification, and variants generated by genetic modification (e.g., pathogens modified in a laboratory by recombinant DNA methods).

As used herein, the term “common cold virus” refers to a virus generally recognized as causing, or being associated with, the symptoms of a common cold. The symptoms of acute nasopharyngitis include sneezing, a runny nose, nasal obstruction or stuffiness or congestion, sore or itchy throat, cough, hoarseness, and mild general symptoms such as headache, fever, chilliness and a general feeling of being unwell. More than 200 viruses are known to cause such symptoms, with the most common being those belonging to the genera of coronavirus, picornavirus, rhinovirus, coxackievirus and adenovirus. Other genera of viruses include parainfluenza virus, respiratory syncytial virus and enterovirus. The term further includes the naturally-occurring form of the viruses (e.g., wild-type); naturally-occurring variants of the viruses; and variants generated in the laboratory, including variants generated by selection, variants generated by chemical modification, and variants generated by genetic modification (e.g., viruses modified in a laboratory by recombinant DNA methods).

As used herein, the term “treatment” refers to any and all uses which remedy a disease state or symptoms, prevent the establishment of disease, or otherwise prevent, hinder, retard, or reverse the progression of disease or other undesirable symptoms in any way whatsoever.

As used herein, the term “comprising” means “including, but not necessarily solely”. Variations of the word “comprising”, such as “comprise” and “comprises”, have correspondingly varied meanings. Thus, for example, a composition “comprising” X may consist exclusively of X or may include one or more additional components.

As used herein, the term “about” in the context of concentrations of components of the formulations, typically means +/−5 or 10% of the stated value, more typically +/−4% of the stated value, more typically +/−3% of the stated value, more typically, +/−2% of the stated value, even more typically +/−1% of the stated value, and even more typically +/−0.5% of the stated value.

In the context of this specification, the terms “therapeutically effective amount” and “diagnostically effective amount” include within their meanings a sufficient but non-toxic amount of a compound or composition of the invention to provide the desired therapeutic or diagnostic effect. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a composition comprising novel compounds capable of inhibiting or treating the symptoms associated with Severe Acute Respiratory Syndrome (SARS) or acute nasopharyngitis (the common cold) in a patient will now be disclosed.

The inventors have surprisingly found that the active compounds as disclosed herein exhibit inhibitive activity to SARS CoV 3CL proteinase in vitro. Accordingly, the active compounds disclosed herein can be used as medicinal agents in the treatment of SARS afflicted patients and for patients afflicted with the acute nasopharyngitis virus.

### The Active Compounds

The active compounds may be represented by the following general formula (I):

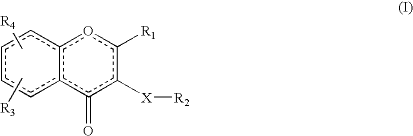

or a pharmaceutically acceptable salt thereof, wherein

[symbol] is an optional double bond;

X is independently selected from oxygen (O), nitrogen (N) and sulfur (S);

R₁ is an optionally substituted aromatic group;

R₂ is selected from the group consisting of straight or branched alkyl, alkenyl, alkynyl, cycloalkyl, lower cycloalkenyl, heterocyclic groups, aromatic groups, heteroaromatic groups, aralkyls, and glycosyl; and

R₃ and R₄ are independently selected from the group consisting of hydrogen (H), hydroxyl, nitro, amino, cyano, imdide, thiol, alkyl, alkenyl, alkynyl, alkoxy, haloalkyl, optionally substituted cycloalkyl, optionally substituted cycloalkenyl, aralkyl, and glycosyl.

In one embodiment, the active compounds are represented by the formula (IA)

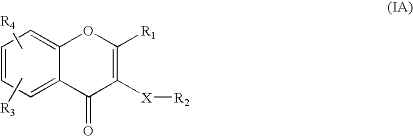

In one embodiment, R₂ is selected from the group consisting of lower alkyl, lower alkenyl, lower alkynyl, lower cycloalkyl, and lower cycloalkenyl. In one embodiment, R₂ is selected from the group consisting of —CH₃, —CH₂CH₃, —CH₂CH₂CH₃, —CH₂CH₂, —CH₂CH₂CH₂, —CH₂CHCH₃, —CHCH₂CH₃, —CCCH₃, —CH₂CCH, —CHCH₂CH₂, and —CHCH₂CH₂CH₂.

In one embodiment, R₃ and R₄ are independently selected from the group consisting of lower alkyl, lower alkenyl, lower alkynyl and lower alkoxy. In one embodiment, R₃ and R₄ are independently selected from the group consisting of —CH₃, —CH₂CH₃, —CH₂CH₂CH₃, —CH₂CH₂—, —CH₂CH₂CH₂, —CH₂CHCH₃, —CHCH₂CH₃, —CCCH₃, —CH₂CCH, —CHO, —COCH₃, and —CH₂CHO.

In one embodiment, X is oxygen.

In one embodiment, R₁ is phenyl radical. In one embodiment, R₁ is a hydroxyl-substituted phenyl radical. In one embodiment, R₁ has the following structure (II):

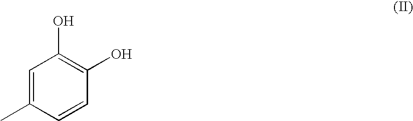

In one embodiment, R₁ is biphenyl radical. In one embodiment, R₁ is a biphenyl heteroatom radical. In one embodiment, R₁ has the following structure (III):

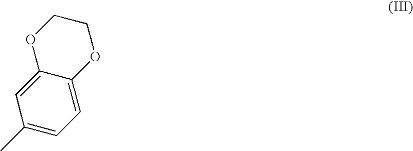

In one embodiment, at least one of the R₃ and R₄ are independently hydroxyl. In one embodiment, R₃ is hydrogen and R₄ is hydroxyl. In another embodiment, R₃ is hydroxyl and R₄ is hydrogen.

In one embodiment, the active compounds are represented by the general formula (IV):

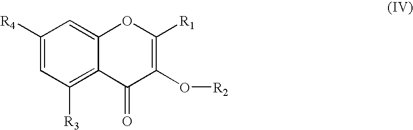

In one embodiment, the active compounds are represented by the general formula (V):

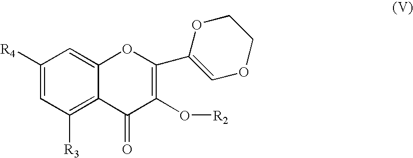

In one embodiment, the active compounds are represented by the general formula (VI):

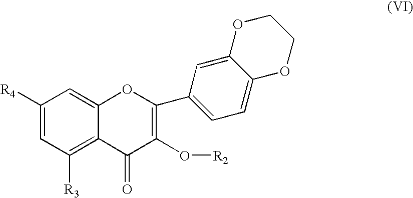

In one embodiment, the active compounds are represented by the general formula (VII):

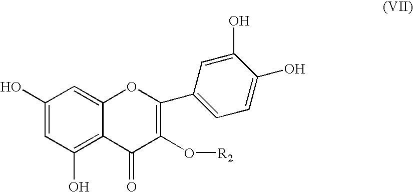

In one embodiment, the active compounds are represented by the general formula (VIII):

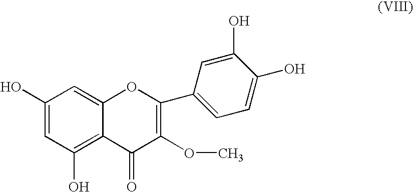

### Exemplary Active Compounds

2-(3,4-dihydroxyphenyl)-5,7-dihydroxy-3-methoxy-4H-chromen-4-one;
2-(3,4-dihydroxyphenyl)-7-hydroxy-5-methyl-3-propoxy-4H-chromen-4-one;
2-(3,5-dihydroxyphenyl)-7-hydroxy-5-methyl-3-propoxy-4H-chromen-4-one;
3-(cyclopropyloxy)-2-(3,4-dihydroxyphenyl)-7-hydroxy-5-methyl-4H-chromen-4-one;
3-(cyclobutyloxy)-2-(3,4-dihydroxyphenyl)-7-hydroxy-5-methyl-4H-chromen-4-one;
3-(cyclopentyloxy)-2-(3,4-dihydroxyphenyl)-7-hydroxy-5-methyl-4H-chromen-4-one;
2-(3,4-dihydroxyphenyl)-5,7-dihydroxy-3-β-L-fucopyranosyloxy-4H-chromen-4-one;
2-(3,4-dihydroxyphenyl)-5,7-dihydroxy-3-β-D-arabinopyranosyloxy-4H-chromen-4-one;
2-(3,4-dihydroxyphenyl)-5,7-dihydroxy-3-β-D-galactopyranosyloxy-4H-chromen-4-one;
2-(3,4-dihydroxyphenyl)-5,7-dihydroxy-3-β-D-glucopyranosyloxy-4H-chromen-4-one;
2-phenyl-3-β-D-galactopyranosyloxy-7-hydroxy-4H-chromen-4-one;
2-(2,3-dihydro-benzo[1,4]dioxin-6-yl)-3-β-D-galactopyranosyloxy-7-hydroxy-4H-chromen-4-one;
3-β-D-galactopyranosyloxy-7-Hydroxy-2-(4-methoxy-naphthalen-1-yl)-4H-chromen-4-one;
2-(2,3-Dihydro-benzo[1,4]dioxin-6-yl)-3-([1,3]dioxolan-2-ylmethoxy)-7-hydroxy-4H-chromen-4-one;
3-Cycloheptyloxy-2-(3,4-dihydroxy-phenyl)-7-hydroxy-4H-chromen-4-one;
2-2,3-Dihydro-benzo[1,4]dioxin-6-yl)-7-hydroxy-3-(2-oxo-tetrahydro-furan-3-yloxy)-4H-chromen-4-one; and
2-(3-fluoro-4-chloro-phenyl)-3-β-D-galactopyranosyloxy-7-Hydroxy-4H-chromen-4-one.

Since the disclosed active compounds may have asymmetric carbon centers, they can be present in the form of racemate, diastereomers or mixtures thereof. Therefore, the present invention also includes all these isomers and their mixtures.

### Synthesis of Active Compounds

The compound of the formula (I) may be prepared by a process described in the following schema.

### Schema I—Synthesis of Active Compounds (I_{A})

Firstly, Quercitin, a known flavanoid, having the following formula (IXa) is reacted with dichlorodiphenylmethane to protect the ortho-dihydroxyl group of the 2-phenyl moiety as represented by reaction (I):

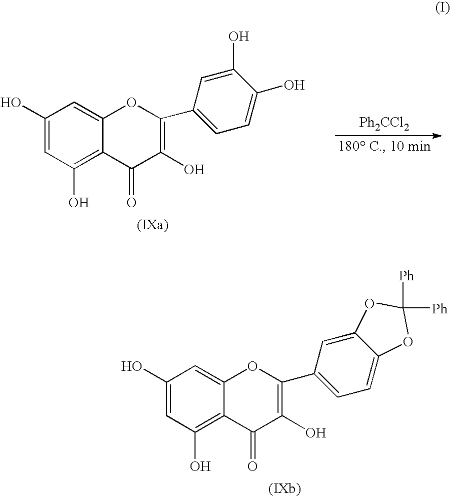

The compound of formula (IXb) then undergoes alkylation as represented by reaction (II):

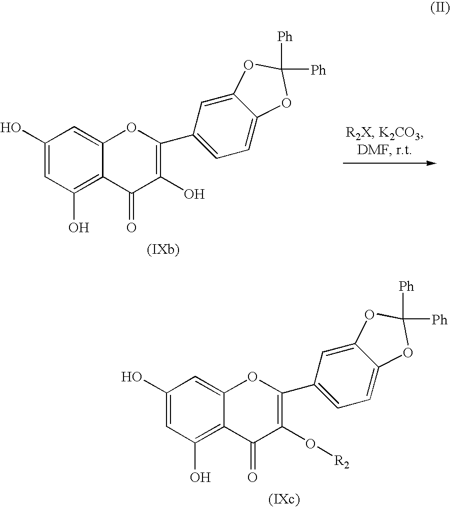

wherein, R₂ is as described above and X is a halide.

Compound (IXc) is then converted to the corresponding deprotected compound (IXd) by catalytic hydrogenation as represented by reaction (III):

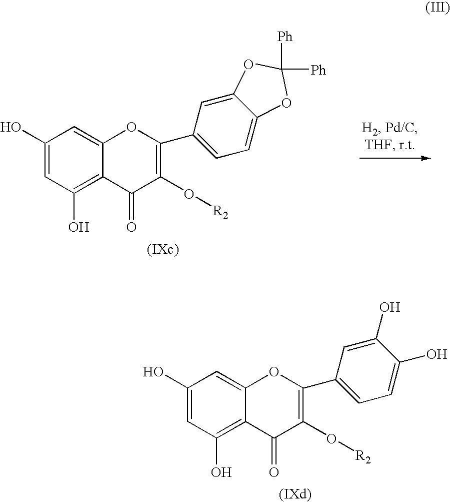

### Schema II—Synthesis of Active Compounds (I_{B})

Firstly, the compound of formula (Xa) is mono-benzylated selectively to give compound Xb as represented by reaction (I):

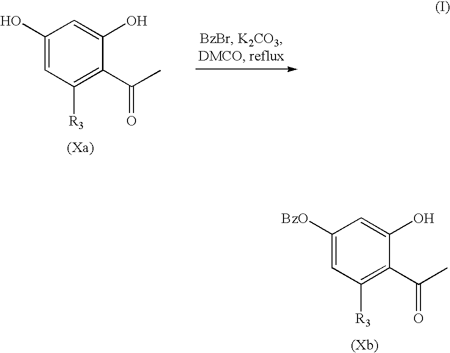

wherein R₃ is as described above.

The compound of formula (Xb) then undergoes AFO (Algar-Flynn-Oyamada) reaction (J. Med. Chem. 2000, 43, 3752-3760) as represented by reaction (II):

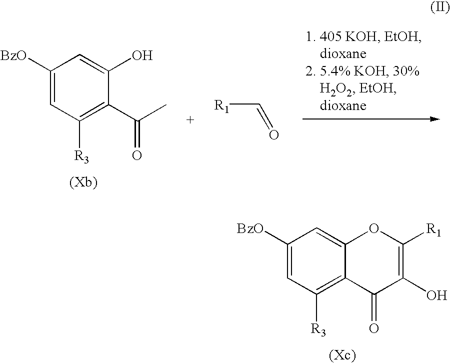

wherein R₁ and R₃ are as described above.

Similar to the synthesis of compound IXb to compound IXd, compound Xe is synthesized as represented by reaction (III):

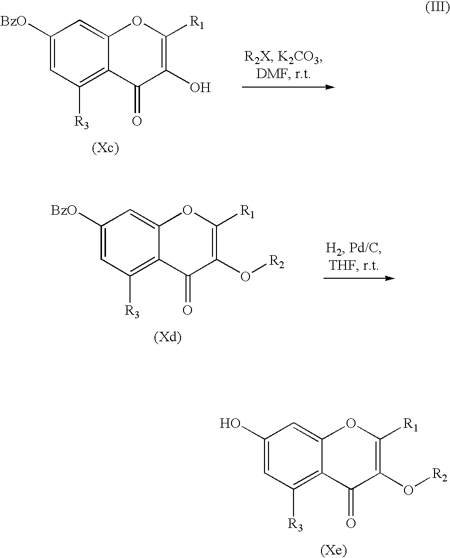

wherein R₁, R₂ and R₃ are as described above.

### Active Compound Salts

In some forms, it may be desirable to formulate the active compounds in pharmaceutically acceptable salt form, generally to improve the solubility and bioavailability and to provide an active drug that may be capable of being assimilated readily.

The active compounds may form pharmaceutically acceptable salts with both organic and inorganic acids. Suitable physiologically tolerated acids for salt formation may be organic and inorganic acids, such as hydrochloric, sulfuric, phosphoric, acetic, citric, oxalic, malonic, salicylic, malic, fumaric, succinic, ascorbic, maleic, methanesulfonic, isethionic, lactic, gluconic, glucuronic, sulfamic, benzoic, tartaric, pamoic, and the like.

The salts may be prepared by contacting a free base form with an equivalent amount of the desired acid in the conventional manner. The free base forms may be regenerated by treating the salt form with a base. For example, dilute aqueous base solutions may be utilized. Dilute aqueous sodium hydroxide, potassium carbonate, ammonia, and sodium bicarbonate solutions may be suitable for this purpose.

The free base forms may differ from their respective salt forms somewhat in certain physical properties such as solubility in polar solvent. Otherwise, the salts may be equivalent to their respective free base forms for purposes of the invention.

The active compounds may exist in unsolvated as well as solvated forms, including hydrated forms. Such salt forms of the active compound may be provided or mixed prior to use with a physiologically acceptable solvent such as water or ethanol.

### Pharmaceutically Acceptable Carriers

The active compounds disclosed herein may include a conventional pharmaceutical carrier or excipient, and in addition, may include other medicinal agents, pharmaceutical agents, carriers, adjuvants, etc. Examples of suitable pharmaceutical carriers include phosphate buffered saline solutions, water, emulsions, such as oil/water emulsions, various types of wetting agents, sterile solutions etc. Other suitable pharmaceutical carriers are described in Remington's Pharmaceutical Sciences, Mack Publishing Company, a standard reference text in this field, or in U.S. Pharmacopeia National Formulary, 1857-1859, (1990). Compositions comprising such carriers may be formulated by conventional methods.

### Mode of Administration

Administration of the active compounds disclosed herein, in pure form or in an appropriate pharmaceutical composition, may be carried out via any of the acceptable modes of administration or pharmaceutically acceptable means of delivery. The modes of administration and pharmaceutically acceptable means of delivery may include oral administration or delivery in the form of solid, semi-solid, lyophilized powder, or liquid dosage forms. The dosage forms may include tablets, suppositories, pills, soft elastic and hard gelatin capsules, powders, solutions, suspensions, or aerosols, or the like, preferably in unit dosage forms suitable for simple administration of precise dosages.

Oral administration of the disclosed active compounds may be effected by preparing a mixture of the disclosed active compounds with an inert diluent or with an assimilable edible carrier, or they may be enclosed in hard or soft shell gelatin capsules, or they may be compressed into tablets, or they may be incorporated directly with the food of the diet. For oral therapeutic administration, the disclosed active compounds may be incorporated with excipients and used in the form of ingestible tablets, buccal tables, troches, capsules, elixirs, suspensions, syrups, wafers, and the like. Such compositions and preparations may contain the active compounds in an amount by weight percent selected from the group consisting of about 0.1% to about 70%, about 0.5% to about 65%, about 1% to about 60%, about 2% to about 55% and about 3% to about 50%.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin or a flavoring agent, such as peppermint, oil of wintergreen, or cherry flavoring may be added. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent, methyl and propylparabens as preservatives, a dye and flavorings, such as cherry or orange flavor. Apparently, any material used in preparing any dosage unit form should be pharmaceutically pure and substantially non-toxic in the amounts employed. In addition, the active compounds may be incorporated into sustained-release preparation and formulations.

The active compounds disclosed herein may be administered parenterally or intraperitoneally. Solutions of the disclosed active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use may include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating, such as lecithin, by the maintenance of the required particle size in the case of dispersion and by the use of surfactants. The prevention of the action of microorganisms may be brought about by various antibacterial and antifungal agents, for example, parabens, chlorobutanol, phenol, sorbic acid, thimerosal, and the like.

### Administration Dosage

The active compounds in pharmaceutically acceptable form may be administrated in a therapeutically effective amount which will vary depending upon a variety of factors including the activity of the specific compounds employed; the metabolic stability and length of action of the compounds; the age, body weight, general health, sex and diet of the patient; the mode and time of administration; the rate of excretion; the drug combination; the severity of the particular disease states; and the patient undergoing treatment.

### Active Compounds as a Therapeutic Agent

It has been surprisingly found that the active compounds of general formula (I) may be capable of inhibiting both the 3CL proteinases (3CL M^{pro}) of SARS-CoV and common cold virus (HCoV-229E antigenic type). Hence, the disclosed active compounds may be capable of inhibiting the viral replication of both SARS-CoV and the acute nasopharyngitis virus. The active compounds may be envisaged as being useful in the treatment of patients suffering from SARS and the acute nasopharyngitis.

### EXAMPLES

Non-limiting embodiments of the disclosed active compounds will be further described in greater detail by reference to specific Examples, which should not be construed as in any way limiting the scope of the invention.

The reagents (chemicals) were purchased from Lancaster, Acros and Shanghai Chemical Reagent Company, and used without further purification unless otherwise stated. Analytical thin-layer chromatography (TLC) was HSGF 254 (150-200 pm thickness, Yantai Huiyou Company, China). Silica gel used in Column chromatography is 200-300 mesh (Shanghai Chemical Reagent Company, China).

Yields were not optimized. Melting point (Mp) was measured in a capillary tube on a SGW X-4 melting point apparatus without correction. Nuclear magnetic resonance (NMR) spectra were given on a Brucker AMX-400 NMR (IS as TMS). Chemical shifts were reported in parts per million (ppm, δ) downfield from tetramethylsilane. Proton coupling patterns were described as singlet (s), doublet (d), triplet (t), quartet (q), multiplet (m), and broad (br). Low- and high-resolution mass spectra (LRMS and HRMS) were given with electric and electrospray ionization (EI and ESI) produced by Finnigan MAT-95 and LCQ-DECA spectrometer.

### Preparation of Active Compounds

### Example 1

### Step 1—Preparation of 2-(2,2-diphenyl-benzo[1,3]dioxolan-5-yl)-3,5,7-trihydroxy-benzopyran-4-one (IXb)

A mixture of quercetin IXa (5 g, 15 mmol) and Ph₂CCl₂ (8.5 mL, 45 mmol) in a 50 mL round bottom flask was stirred at 180° C. for 10 min. The reaction mixture was purified by flash chromatography on silica gel and eluted with a mixture of EtOAc/petroleum ether (1:4, v/v) to afford IXb (2.5 g, 37%) as a yellow solid: Mp 239-240° C., ¹H-NMR (DMSO-d6) δ: 6.22 (1H, d), 6.50 (1H, d), 7.26 (1H, d), 7.44-7.60 (10H, m), 7.80-7.83 (2H, m).

### Step 2: Preparation of 2-(2,2-diphenyl-benzo[1,3]dioxolan-5-yl)-3-O-β-D-tetraacetylglucopyranosyl-5,7-dihydroxy-benzopyran-4-one (IXc-1)

A mixture of IXb (0.3 g, 0.64 mmol), acetobromo-α-D-glucose (0.4 g, 0.97 mmol) and K₂CO₃ (0.12 g, 0.9 mmol) in DMF (5 mL) was stirred at room temperature of about 21° C. for 10 h under argon. The reaction mixture was poured into water (50 mL) and extracted with EtOAc (3×30 mL). The combined organic layer was washed with water, dried with anhydrous MgSO₄, filtered and condensed under reduced pressure. The crude product obtained was purified by flash chromatography on silica gel and eluted with a mixture of EtOAc/petroleum ether (1:4, v/v) to afford IXc-1 (0.16 g., 31%). MS-ESI 797 [M+H]+.

### Step 3: Quercetin-3-O-β-D-glucopyranoside (IA-1)

A mixture of IXc-1 (160 mg, 0.2 mmol), 10% palladium on charcoal (20 mg), and EtOH (20 mL) was stirred at 25° C. for 24 h in an atmosphere of hydrogen. The catalyst was filtered and the filtrate was concentrated under reduced pressure to dryness. The crude product obtained was purified by flash chromatography on silica gel and eluted with a mixture of EtOAc/petroleum ether (1:2, v/v) to afford 2-(3,4-dihydroxyphenyl)-3-O-β-D-tetraacetylglucopyranosyl-5,7-dihydroxy-benzopyran-4-one (IXd-1) 62 mg. MS-ESI 633 [M+H]⁺.

To a solution of IXd-1 (50 mg, 0.079 mmol) in methanol (10 mL), NaOMe (5 mg, 0.09 mmol) was added and the resultant solution was stirred at room temperature of about 21° C. for 1 h. The solution was neutralized by passage down a Dowex 50 (H⁺) ion-exchange resin. The resin was filtered and the filtrate was concentrated to afford IA-1 (30 mg, 81%). Mp 172-174° C. ¹H-NMR (DMSO-d6) δ: 3.1-3.8 (7H, m), 5.49 (1H, d), 6.22 (1H, d) 6.43 (1H, d), 6.87 (1H, d), 7.61 (2H, m). MS-ESI 465 [M+H]+, HRMS (ESI) m/z calculated C₂₁H₂₁O₁₂ [M+H]+ 465.1033, found 465.1067.

### Example 2

### Quercetin-3-O-β-D-galactopyranoside (IA-2)

Quercetin-3-O-β-D-galactopyranoside (IA-2) was prepared by the same procedure and materials as Example 1 but using acetobromo-α-D-pyranogalactose to replace acetobromo-α-D-pyranoglucose in step 2.

75 mg-yellow solid was obtained with Mp 187-190° C. The overall yield was 45.7% (last two steps). 1H-NMR (400 Hz, DMSO-d6) δ: 3.20-3.62 (7H, m), 5.37 (1H, d), 6.19 (1H, d), 6.40 (1H, d), 6.82 (1H, d), 7.51 (1H, d), 7.66 (1H, d). MS-ESI 465 [M+H]+, HRMS (ESI) m/z calcd C21H21O12 [M+H]+465.1053, found 465.1067.

### Example 3

### Quercetin-3-O-β-D-arabinopyranoside (IA-3)

Quercetin-3-O-β-D-arabinopyranoside (IA-3) was prepared by the same procedure and materials as Example 1 but using acetobromo-α-D-pyranoarabinose to replace acetobromo-α-D-pyranoglucose in step 2.

37 mg yellow solid was obtained with Mp 41-44° C. The overall yield was 40.7% (last two steps). 1H-NMR (400 Hz, DMSO-d6) δ: 3.20-3.80 (6H, m), 5.30 (1H, d), 6.23 (1H, d), 6.44 (1H, d), 6.87 (1H, d), 7.66 (1H, d), 7.69 (1H, d). MS-ESI 435 [M+H]+, HRMS (ESI) m/z calculated C₂₀H₁₉O₁₁ [M+H]+ 435.0927, found 435.0909.

### Example 4

### Quercetin-3-O-β-D-fucopyranoside (IA-4)

Quercetin-3-O-β-D-fucopyranoside (IA-4) was prepared by the same procedure and materials as Example 1 but using acetobromo-α-D-pyranofucose to replace acetobromo-α-D-pyranoglucose in step 2.

63 mg yellow solid was, obtained with Mp 172-174° C. The overall yield is 38.7% (last two steps). 1H-NMR (400 Hz, DMSO-d6) δ: 1.02 (3H, d), 3.30-3.60 (5H, m), 5.36 (1H, d), 6.23 (1H, d), 6.44 (1H, d), 6.86 (1H, d), 7.56 (1H, d), 7.71 (1H, d). MS-ESI 449 [M+H]+, HRMS (ESI) m/z calcd C21H21O11 [M+H]+ 449.1084, found 449.1087.

### Example 5

### Step 1: Synthesis of 3′,4′,7-tribenzyloxy-flavonol (VI-1)

1.21 g of 4-benzyloxy-2-hydroxylacetophenone and 1.59 g of 3,4-dibenzyloxybenzaldehyde were added into 20 ml solution of dioxane:ethanol (3:2).

The reaction mixture was stirred and cooled in an ice bath to about 0° C. before 4 ml 40% KOH was added to the reaction mixture.

The reaction mixture was then stirred for 66 hours at room temperature of about 21° C.

After stirring, the crude product was washed with 125 ml dichloromethane and three times with 40 ml water, respectively before being dried, filtered and concentrated under reduced pressure to provide an oil mixture (about 10 mL).

The above oil mixture was added to 16 ml 5.4% NaOH, 17 ml dioxane and 47 ml absolute ethane alcohol while being stirred in an ice bath so that the temperature of the reactant solution was about 0° C.

2.1 ml 30% H₂O₂ was dropped into the reaction mixture, which was stirred for 2 hours at 0° C. The reaction mixture was then stirred for another 12 hours at room temperature of about 21° C.

The crude product was acidified with 32 ml 2M HCl, filtered under reduced pressure and washed with water and ethanol, respectively to yield 2.3 g of a yellow solid product (VI-1). The overall yield is 42.7% (two steps). 1H-NMR (400 Hz, CDCl3) δ: 5.17 (2H, s), 5.25 (4H, s), 7.06 (3H, m), 7.25-7.53 (15H, m), 7.78 (1H, d), 7.90 (1H, s), 8.13 (1H, d).

### Step 2: 3-O-cyclohexyl-3′,4′,7-tribenzyloxy-flavonol (VII-1)

100 mg VI-1, 150 mg bromocyclohexane and 50 mg potassium carbonate were added into 5 ml DMF. The reaction mixture was warmed to 100° C. and stirred for 24 hours under Ar atmosphere before being cooled to room temperature of about 21° C.

The reaction mixture was poured into water (50 mL) and extracted with EtOAc (3×30 mL). The organic layer was dried with anhydrous MgSO₄, and the solvent removed by distillation under reduced pressure. The reminder was separated by column chromatography and washed with petrolic ether:EtOAc (8:1) to provide 90 mg yellow solid (yield 75.0%). 1H-NMR (400 Hz, DMSO-d6) δ: 1.06-1.40 (6H, m), 1.60 (2H, m), 1.80 (2H, m), 4.24 (1H, m), 5.17 (2H, s), 5.23 (2H, s), 5.26 (2H, s), 6.92 (1H, d), 7.05 (2H, d), 7.30-7.50 (15H, m), 7.70 (1H, d), 7.84 (1H, d), 8.16 (1H, d).

### Step 3: 3-O-cyclohexyl-3′,4′,7-trihydroxy-flavonol (IB-1)

80 mg VII-1 and 20 mg Pd/C (10%) were added into 20 ml absolute ethane alcohol.

The reaction mixture was stirred for 24 hours at room temperature of about 21° C. The reactant product was filtered and then concentrated under reduced pressure to about 2 ml, which was separated using column chromatography and washed with petrolic ether:EtOAc (2:1) to provide 17 mg yellow solid (yield 36.2%). Mp 98-100° C. 1H-NMR (400 Hz, DMSO-d6) δ: 1.06-1.40 (6H, m), 1.60 (2H, m), 1.80 (2H, m), 4.24 (1H, m), 6.85-6.92 (3H, m), 7.47 (1H, d), 7.57 (1H, d), 7.90 (1H, d). MS-EI 368 (M+), 286 (100%). HRMS (EI) m/z calcd C21H20O6 (M+) 368.1258, found 368.1260.

### Example 6

### 3-[2-(2,5-dimethoxyphenyl)-2-oxo-ethoxy]-3′,5′,7-trihydroxy-flavonol (IB-2)

3-[2-(2,5-dimethoxyphenyl)-2-oxo-ethoxy]-3′,5′,7-trihydroxy-flavonol (IB-2) was prepared by the same procedure and materials as Example 5. However, α-bromo-2,5-dimethoxyacetophenone was used in place of bromocyclohexane in step 2.

25 mg yellow solid was obtained with Mp 138-140° C. The yield is 25.0%. 1H-NMR (400 Hz, DMSO-d6) δ: 3.72 (3H, s), 3.79 (3H, s), 5.33 (2H, s), 6.84-6.91 (4H, m), 7.16 (2H, m), 7.26 (1H, d), 7.59 (2H, m), 7.88 (1H, d). MS-ESI 46.5 [M+H]+. HRMS (ESI) m/z calcd C25H21O9 [M+H]+ 465.1186, found 465.1164.

### Example 7

### 3-O-cycloheptyl-3′,4′,7-trihydroxy-flavonol (IB-3)

3-O-cycloheptyl-3′,4′,7-trihydroxy-flavonol (IB-3) was prepared by the same procedure and materials as Example 5. However, bromocycloheptane was used in place of bromocyclohexane in step 2.

110 mg yellow solid was obtained with Mp 68-71° C. The yield was 75.0%. 1H-NMR (400 Hz, DMSO-d6) δ: 1.20-1.60 (8H, m), 1.87 (4H, m), 4.45 (1H, m), 6.85-6.91 (3H, m), 7.45 (1H, d), 7.57 (1H, s), 7.89 (1H, d). MS-EI 382 (M+), 286 (100%). HRMS (EI) m/z calcd C22H22O6 (M+) 382.1425, found 382.1416.

### Example 8

### 3-(2,3-dihydro-benzo[1,4]dioxan-2-yl-methoxy)-3′,5′,7-trihydroxy-flavonol (IB-4)

3-(2,3-dihydro-benzo[1,4]dioxan-2-yl-methoxy)-3′,5′,7-trihydroxy-flavonol (IB-4) was prepared by the same procedure and materials as Example 5. However, 2-bromomethyl-2,3-dihydro-benzo[1,4]dioxane was used in place of bromocyclohexane in step 2.

90 mg yellow solid was obtained. 1H-NMR (400 Hz, DMSO-d6) δ: 2.38 (3H, s), 2.45 (3H, s), 4.67 (2H, s), 5.21 (2H, s), 7.25-7.46 (7H, m), 7.55 (1H, s), 7.84 (1H, d), 7.94 (1H, s), 8.47 (1H, d), 8.61 (1H, s). MS-ESI 460 [M+H]+.

### Example 9

### 3-(2-oxo-tetrahydrofuran-3-yl-oxy)-3′,5′,7-trihydroxy-flavonol (IB-5)

3-(2-oxo-tetrahydrofuran-3-yl-oxy)-3′,5′,7-trihydroxy-flavonol (IB-5) was prepared by the same procedure and materials as Example 5. However, 3-bromo-dihydrofuran-2-ketone was used in place of bromocyclohexane in step 2.

29 mg yellow solid was obtained. 1H-NMR (400 Hz, DMSO-d6) δ: 4.69 (2H, s), 5.20 (2H, s), 7.31-7.46 (4H, m), 7.55 (2H, d), 7.73 (1H, d), 7.84 (1H, d), 7.94 (1H, s), 8.06 (1H, d), 8.48 (1H, d), 8.62 (1H, s). MS-ESI 557 [M+H]+.

### Example 10

### 2-(2,3-dihydro-benzo[1,4]dioxan-6-yl)-3-hydroxy-7-benzyloxy-benzopyran-4-one (VI-2)

2-(2,3-dihydro-benzo[1,4]dioxan-6-yl)-3-hydroxy-7-benzyloxy-benzopyran-4-one (VI-2) was prepared by the same procedure and materials as Example 5. However, 2,3-dihydro-benzo[1,4]dioxane-6-formaldehyde was used in place of 3,4-dibenzyloxybenzaldehyde in step 1.

0.6 g yellow solid was obtained. The overall yield is 15.5% (two steps). MS-ESI 387 [M+H]+.

### Example 11

### 2-(2,3-dihydro-benzo[1,4]dioxan-6-yl)-3-O-cyclohexyl-7-benzyloxy-benzopyran-4-one (VII-2)

VII-2 was prepared by the same procedure and materials as Example 5 but using VI-2 to replace VI-1 in step 2.

200 mg yellow solid was obtained (yield 83.3%). MS-ESI 499 [M+H]+.

### Example 12

### 2-(2,3-dihydro-benzo[1,4]dioxan-6-yl)-3-O-cyclohexyl-7-hydroxy-benzopyran-4-one (IB-6)

100 mg VII-2 and 20 mg Pd/C (10%) were added into 20 ml absolute ethane alcohol. The reaction mixture was stirred for 24 hours at room temperature of about 21° C., filtered and then concentrated under reduced pressure to about 2 ml. The mixture was then separated by column chromatography and washed with petrolic ether:EtOAc (2:1) to yield 55 mg of a yellow solid (yield 69.6%). Mp 137-139° C. 1H-NMR (400 Hz, DMSO-d6) δ: 1.06-1.40 (6H, m), 1.60 (2H, m), 1.80 (2H, m), 4.10 (1H, m), 4.34 (4H, t), 6.94 (2H, m), 7.04 (1H, d), 7.65 (2H, m), 7.92 (1H, d). MS-EI 394 (M+), 312 (100%). HRMS (EI) m/z calculated as C₂₃H₂₂O₆ (M+) 394.1431, found 394.1416.

### Example 13

### Methyl 4-[2-(2,3-dihydro-benzo[1,4]dioxan-6-yl)-7-hydroxy-benzopyran-4-one-3-oxy]-butanoate (IB-7)

Methyl 4-[2-(2,3-dihydro-benzo[1,4]dioxan-6-yl)-7-hydroxy-benzopyran-4-one-3-oxy]-butanoate (IB-7) was prepared by the same procedure and materials as preparing IB-6 in Example 12 above but using methyl-γ-butprate to replace bromocyclohexane.

320 mg yellow solid was obtained (yield 89.6%). Mp 168-170° C. 1H-NMR (400 Hz, DMSO-d6) δ: 1.86 (2H, m), 2.42 (2H, t), 3.56 (3H, s), 3.97 (2H, t), 4.34 (4H, m), 6.92 (2H, m), 7.03 (1H, d), 7.55 (2H, m), 7.91 (1H, d). MS-El 412 (M+), 101 (100%). HRMS (EI) m/z calcd C22H20O8 (M+) 412.1138, found 412.1159.

### Biological Studies

### Example 14

### Binding Activity Assay of Some Compounds and 3CL Proteinase of SARS and Common Cold Virus

The study on the binding property between the compounds of 3-alkoxy substituted-2,5,7-tri substituted benzopyran-4-one and 3CL proteinase was based on SPR (Surface Plasmon Resonance) techniques. The instrument used was Biacore 3000 (Biacore AB, Uppsala, Sweden).

The following studies were undertaken with isolated SARS 3CL proteinase and 3CL proteinase of common cold virus

(1) The Construction of SARS 3CL Proteinase (pQE30-SARS 3CLpro)

Fluorescent substrate Dabcyl-KNSTLQSGLRKE-Edans was synthesized and obtained from Shanghai Sheng Gong Biotechnology Limited Company.

SARS CoV 3CL proteinase was expressed in M15 E. coli using expression plasmid constructed from pQE30 vector and purified through NTA-Ni column chromatography by cellular and molecular biology methods in our laboratory. A method to construct the pQE30-3CL expression plasmid can be seen in Molecular cloning, expressions, purification and mass spectrometric characterization of 3C-like protease of SARS coronavirus by H. Sun et al. Protein Expression and Purification 32 (2003) 302-308.

### (2) Expression and Purification of SARS CoV 3CL Proteinase

pQE30-3CL expression plasmid constructed by our lab was used to transform M15 E. coli. SARS 3CL proteinase was purified by NTA Ni column. A method to express and purify SARS CoV 3CL proteinase can be seen in Molecular cloning, expression, purification and mass spectrometric characterization of 3C-like protease of SARS coronavirus by H. Sun et. al., Protein Expression and Purification, 32 (2003), pages 302 to 308.

Absorbance at 280 nm was measured for the freshly prepared protein by U-2010 spectrophotometer (HITACH Company) and the concentration of protein was calculated. The results were used for the screening of inhibitors and IC50 determination. A particularly useful parameter for enzyme inhibitors is the IC50 value. The IC50 value indicates the concentration of the active compound which inhibits 50% of the enzymatic activity.

### (3) Coupling of 3CL Proteinase

A Biacore 3000 (Biacore AB, Uppsala, Sweden) was used to analyse coupling between the active compounds and the enzyme. After cleaning, the Biacore 3000 table was set to base line using damping fluid of HBS-EP consisting of 10 mM Hepes, 150 mM NaCl, 3 mM EDTA and 0.005% (v/v) surfactant P20, pH 7.4.

0.2 M N-ethyl-N′-dimethylaminopropyl carbodiimide (EDC) and 50 mM N-hydroxysuccinimide (NHS) with 1:1 ratio were automatically mixed and injected to the surface of the chip under the control of Biacore 3000 Software. Then, the chip (CM5 Biacore AB, Uppsala, Sweden) was activated with 5 μL/min for 7 minute.

SARS-CoV 3CL^{pro} was diluted with 10 mM sodium acetate buffer at pH 4.3 to a concentration of 25 μg/mL and immobilized to the surface of sensor chip CM5 at 5 μL/min. Finally, unreacted protease was blocked by injecting 1 M ethanolamine-HCl at pH 8.5 for 7 min. The final coupling amount was 4000 resonance units (RU).

### (4) The Screening of Compounds

The active compounds synthesized in Examples 1 to 13 were dissolved in 100% DMSO, with the final concentration of 10 mM, and then diluted by damping fluid HBS-EP to 1 μM and 10 μM. The final concentration of DMSO was 0.1%.

The binding activity of compounds to SARS 3CL proteinase was determined by the value of RU.

### (5) Dynamic Test

The compounds were diluted to different concentrations (80 μM, 56 μM, 39.2 μM, 27.4 μM, 19.2 μM, 13.4 μM and 9.4 μM) with HBS-EP buffer and injected at a constant flow of 30 μL/min at 25° C. Sensorgrams were processed by automatically subtracting for non-specific bulk refractive index effects. The kinetic parameters (KD) were analyzed using a global data analysis program (BIAevaluation 3.1 software, Biacore AB).

### (6) Experimental Results

The KD values of the compounds (IA-1, IA-3, IA-4, IB-1, IB-3 and IB-4) synthesized as described above were determined for SARS 3CL and HCV 3CL proteinases and the results tabulated in Table 1 below.

TABLE 1

KD (μM)

| No | Name | SARS 3CL | HCV 3CL |
|---|---|---|---|
| 1 | IA-1 | 26.7 | 28.8 |
| 2 | IA-3 | 13.2 | 3.76 |
| 3 | IA-4 | 104 | 5.75 |
| 4 | IB-1 | 33.7 | 3.52 |
| 5 | IB-3 | 33.5 | 1.51 |
| 6 | IB-4 | 16.8 | 2.60 |

It can be seen from the data of Table 1 that the compounds (IA-1, IA-3, IA-4, IB-1, IB-3 and IB-4) couple strongly with the SARS 3CL. The compounds (IA-3, IA-4, IB-1, IB-3 and IB-4) have high affinity with HCV 3CL proteinases. However, IA-1 and IA-4 had weaker affinity with HCV 3CL and SARS 3CL protease than other compounds, respectively. It might suggest that the affinity selectivity change because of the structural alteration.

### (7) IC50 Determination

### (7.1) Experimental Materials

The inhibition activity (IC50) of some of the active compounds to SARS 3CL proteinase was measured by fluorescence resonance energy transfer (FRET). An exemplary FRET method can be seen in Small molecules targeting severe acute respiratory syndrome human coronavirus by C. Wu et al., PNAS, 101:27, (2004) pages 10012 to 10017. The fluorescent substrate for SARS-CoV 3Cl proteinase was synthesized according to the cleavage specificity. (core sequence Leu-Gln-↓-Ser) of the substrate of the proteinase. The amino acid sequence of the substrate was EDANS-Val-Asn-Ser-Thr-Leu-Gln-Ser-Gly-Leu-Arg-Lys-(Dabcyl)-Met. EDANS and Dabcyl are a widely used fluorescent-quenching molecular pair.

The change in florescence intensity of the substrate was measured by TECAN GENios Invitrogen. The change in florescence intensity of substrate relies on the enzymatic activity. If the enzyme can cleave the substrate, the florescence intensity will increase and reach the maximum in the end. The optimal excitation wavelength was 340 nm, and the optimal emission wavelength was 488 nm for an hour.

Different concentrations (100 mM˜20 μM) of stock solution for the active compounds were prepared and mixed with 1 μM 3CL proteinase. The final concentration of the fluorescent substrate was 10 μM. The volume of the solution was 200 μl., and the final concentration of the active compounds was 500 μM˜0.1 μM respectively. The concentrations from 100 mM to 20 μM for the active compounds are used as stock solutions. When they were diluted to 200 μl according to the ratio 1:200, the respective concentrations are from 500 μM to 0.1 μM.

During measurement of IC50 of the active compounds, a blank reference was set using 0.5% DMSO. Multiple wells were used for each sample and the average value was taken for each measurement. With different concentrations of the compound, the inhibition rates of different concentrations were calculated from the comparison of reaction speed between the samples with the compound and the blank samples.

The IC50 of inhibition against 3CL proteinase was calculated by Logistic formulae and Origin software using non-linear fitting. The formulae used was as follows:

$$\frac{A ( I )}{{A}_{0}} = 1 - \frac{1}{1 + {( \frac{I}{{IC}_{50}} )}^{p}}$$

Where:

A(I) is the enzyme activity of the blank reference

A(I) is the enzyme activity of various concentrations of the active compounds

I is the concentration of inhibitors.

### (7.2) Experimental Result

The IC50 results of the active compounds IA-I, IA-2, IA-3, IA-4, and IB-1 are disclosed in Table 2.

TABLE 2

| No | Name | SARS 3CL IC₅₀ (μM) |
|---|---|---|
| 1 | IA-1 | 49.73 |
| 2 | IA-2 | 50.50 |
| 3 | IA-3 | 31.62 |
| 4 | IA-4 | 24.14 |
| 5 | IB-1 | 33.15 |

It can be seen from Table 2 that the active compounds have a high affinity with the SARS 3CL proteinase and thereby exhibit activity in inhibiting the SARS virus.

### Example 15

### Preparation of Oral Tablet for a Series Compounds of 3-alkoxy substituted-2,5,7-tri substituted benzopyran-4-one

Tablets were prepared for oral administration to patients by mixing the following constituents into a homogenous mixture:

125 g active compounds

10 g Vitamin C

40 g Micronized silica gel

10 g Colloidized amylum

4 g Magnesium stearate

The homogenous mixture of above components was formed into 500 tablets by wet method and vacuum drying. Each tablet has 250 mg of active compound. The ‘wet method’ is a known methods to make tablets in the pharmaceutical industry. Firstly., the active compounds, Vitamin C, Micronized silica gel and magnesium stearate are blended and milled to form mixed powder. Secondly, the Colloidized amylum is made into a paste (e.g., in a 1:10 ratio) with water, which is used as “Binder Solution”. Lastly, the binder paste is added into the powder and mixed to form a wet mass from which the tablets are formed.

### Example 16

### Preparation of Oral Solution for a Series Compounds of 3-alkoxy substituted-2,5,7-tri substituted benzopyran-4-one

An oral solution was prepared for oral administration to patients by mixing the following constituents into a homogenous mixture:

100 g Cane sugar

5 g Sodium carboxymethyl cellulose

30 g Tween

500 g Distilled water

The constituents were mixed thoroughly at 60° C. The mixture was then allowed to cool to 20° C. before 10 g Active Compounds and 50 g Vitamin C was added to the mixture to make 1000 ml oral solution.

### Example 17

### Preparation of Capsule for a Series Compounds of 3-alkoxy substituted-2,5,7-tri substituted benzopyran-4-one

A capsule was prepared for oral administration to patients by mixing the following constituents into a homogenous mixture:

125 g active compounds

50 g Vitamin C

10 g Ls-Hydroxypropyl cellulose

40 g Micronized silica gel

25 g Microcrystalline cellulose

The above constituents were mixed in the presence of 70% ethanol used as a humectant and followed by vacuum drying.

4 g of magnesium stearate was added to make 1000 capsules, in which 0.125 g of the active compounds was present in each capsule.

### Example 18

### Preparation of Injection Medicines for a Series Compounds of 3-alkoxy substituted-2,5,7-tri substituted benzopyran-4-one

A medicine was prepared for injection into patients by mixing the following constituents into a homogenous mixture:

300 g active compounds

1350 ml 45% ethanol

1500 g polyoxyethylene castor oil

The homogeneous mixture was refrigerated and then filtered to make into 1000 little bottles. There was 0.3 g of active compounds in each bottle. The solution could be placed into a drip after dilution with 250 ml normal saline.

### Example 19

### Preparation of Effervescent Tablet for a Series Compounds of 3-alkoxy substituted-2,5,7-tri substituted benzopyran-4-one

An effervescent tablet was prepared by mixing two portions, namely acidic portion and basic portion.

### Preparation of the Grain of Acidic Portion

100 g active compounds
450 g Citric acid
50 g Lactose

### Preparation of the Grain of Basic Portion

390 g NaHCO₃
40 g Lactose
10 g Aspartame
4 g orange essence

The homogeneous mixture of the two portions was separated into 250 parts forming about 4 g per part and 0.4 g active compound in each part.

### APPLICATIONS

The disclosed active compounds have exhibited the ability to substantially bind with, and inhibit, both 3CL proteinase of SARS and viruses associated with acute nasopharyngitis.

Accordingly, the disclosed active compounds have exhibited substantial anti-SARS activity and anti-acute nasopharyngitis activity. Accordingly, the disclosed active compounds are useful as medicines to treat a patient suffering from SARS, acute nasopharyngitis and other related diseases.

The disclosed active compounds are relatively easy to synthesize from readily available commercial materials.

The disclosed active compounds have very low toxicity to humans and therefore are ideal for use in medicine, particularly for use in medicines for treating, or inhibiting patients suffering from viral infections such as those associated with SARS and acute nasopharyngitis.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A pharmaceutical composition comprising a compound represented by the following formula (I): 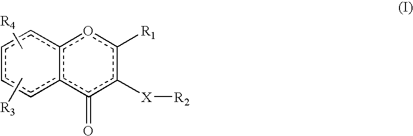
or a pharmaceutically acceptable salt thereof, wherein [symbol]
is an optional double bond;
X is independently selected from oxygen (O), nitrogen (N) and sulfur (S);
R₁ is an optionally substituted aromatic group;
R₂ is selected from the group consisting of straight or branched alkyl, alkenyl, alkynyl, cycloalkyl, lower cycloalkenyl, aromatic groups, heteroaromatic groups, aralkyls, and glycosyl; and heterocyclic groups wherein the heteroatom is selected from the group consisting of N, NH and S; and
R₃ and R₄ are independently selected from the group consisting of hydrogen (H), hydroxyl, nitro, amino, cyano, imdide, thiol, alkyl, alkenyl, alkynyl, alkoxy, haloalkyl, optionally substituted cycloalkyl, optionally substituted cycloalkenyl, aralkyl, and glycosyl.

2. A pharmaceutical composition as claimed in claim 1, wherein the compound is represented by the formula (IA)

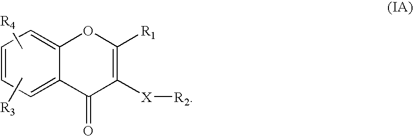

3. A pharmaceutical composition as claimed in claim 1, wherein R₂ is selected from the group consisting of lower alkyl, lower alkenyl, lower alkynyl, lower cycloalkyl, and lower cycloalkenyl.

4. A pharmaceutical composition as claimed in claim 1, wherein R₃ and R₄ are independently selected from the group consisting of lower alkyl, lower alkenyl, lower alkynyl and lower alkoxy.

5. A pharmaceutical composition as claimed in claim 1, wherein X is oxygen.

6. A pharmaceutical composition as claimed in claim 1, wherein R₁ is a phenyl radical.

7. A pharmaceutical composition as claimed in claim 6, wherein R₁ is a hydroxyl-substituted phenyl radical.

8. A pharmaceutical composition as claimed in claim 1, wherein R₁ has the following structure (II):

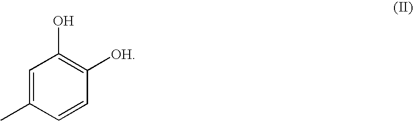

9. A pharmaceutical composition as claimed in claim 1, wherein R₁ is a biphenyl radical.

10. A pharmaceutical composition as claimed in claim 9, wherein R₁ is a biphenyl heteroatom radical.

11. A pharmaceutical composition as claimed in claim 1, wherein R₁ has the following structure (III):

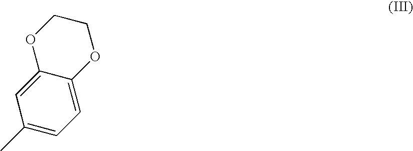

12. A pharmaceutical composition as claimed in claim 1, wherein at least one of R₃ and R₄ are independently hydroxyl.

13. A pharmaceutical composition as claimed in claim 1, wherein R₃ is hydrogen and R₄ is hydroxyl.

14. A pharmaceutical composition as claimed in claim 1, wherein R₃ is hydroxyl and R₄ is hydrogen.

15. A pharmaceutical composition as claimed in claim 1, wherein the compound is represented by the general formula (IV):

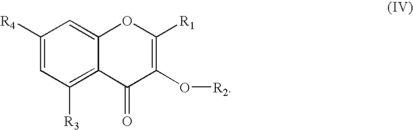

16. A pharmaceutical composition as claimed in claim 1, wherein the compound is represented by the general formula (V):

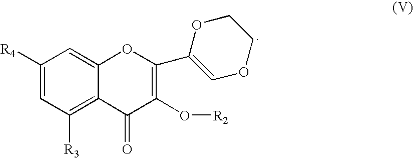

17. A pharmaceutical composition as claimed in claim 1, wherein the compound is represented by the general formula (VI):

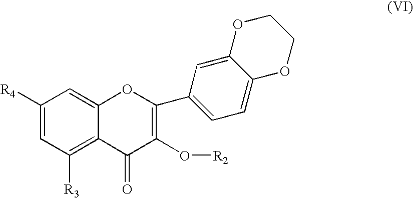

18. A pharmaceutical composition as claimed in claim 1, wherein the compound is represented by the general formula (VII):

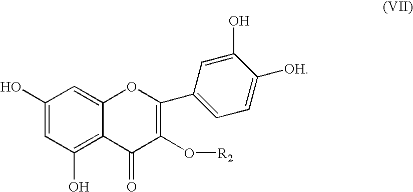

19. A pharmaceutical composition as claimed in claim 1, wherein the compound is represented by the general formula (VIII):

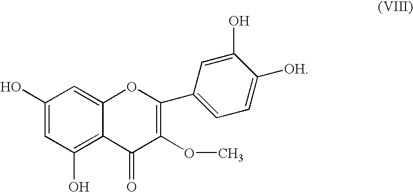

20-28. (canceled)
